(19) United States
(12) Patent Application Publication
(10) Pub. No.: US 2011/0229927 A1
(43) Pub. Date: Sep. 22, 2011

(54) SAMPLE PORT OF A CELL CULTURE SYSTEM

(75) Inventors: Jacob Mollenbach Larsen, Copenhagen NV (DK); Ulrichü Krühne, Copenhagen V (DK)

(73) Assignee: SMART BIOSYSTEMS APS, Copenhagen NV (DK)

(21) Appl. No.: 13/056,860

(22) PCT Filed: Jun. 18, 2009
(86) PCT No.: PCT/DK09/50133

§ 371 Date: May 20, 2011

(30) Foreign Application Priority Data
Aug. 1, 2008 (DK) PA 2008 01062

(51) Int. Cl. C12Q 1/02; C12M 3/00

(52) U.S. Cl. 435/29; 435/287.1; 435/287.2; 435/288.7

(57) ABSTRACT

Disclosed herein is a cell culturing system comprising a culturing chamber for culturing a biological cell in a growth medium and a sensor for measuring a signal in the spent growth medium, wherein the culturing chamber is provided in a mesoscale bioreactor platform with an inlet opening for an influent stream of growth medium and a outlet opening for an effluent stream of spent growth medium, said outlet opening, said spent growth medium being in fluid communication with a sample port for releasable adoption of the sensor. Furthermore, a method of measuring an effluent stream of spent growth medium from a culturing chamber in the cell culturing system is disclosed.

### FIELD OF THE INVENTION

The present invention relates to a cell culturing system for culturing a biological cell and a sensor for measuring a signal in the spent growth medium, wherein a culturing chamber is provided in a mesoscale bioreactor platform with an inlet opening and a outlet opening in fluid communication with a sample port for releasable adoption of the sensor. The invention further relates to a method of measuring an effluent stream from a culturing chamber in the cell culturing system; the measured signal may be used for adjusting the conditions in the culturing chamber.

### PRIOR ART

The procedures currently employed in in vitro fertilisation (IVF) for embryo culture rely on culturing the embryos in Petri-dishes under static conditions. Such methodology is labour-intensive as changes of growth media require a large degree of manual handling. Manual handling always introduces a risk of contamination, and moreover the static conditions do not provide much resemblance with in vivo conditions, as it is difficult to meet the changing needs of an embryo. In contrast to the current-day in vitro static conditions an embryo in vivo is exposed to a constantly changing environment, and the requirements of an embryo in one stage of development may be very different to those in another stage of development. The conditions existing in vivo at one stage of development may even be harmful to an embryo at a later stage of development.

Some of the disadvantages of the static-based Petri-dish culturing system may be circumvented by culturing the embryo in a culturing system capable of perfusing the embryo with a growth medium appropriate for its developmental stage. Such a system should be sized appropriately to match the size of the embryo and to more closely resemble the conditions existing in vivo. Furthermore, it is important to work in small scale to minimise the consumption of expensive growth media typically required by such mammalian cells.

Many so-called microfluidic devices have now been described for conducting various types of analysis or for culturing cells. These devices are often created using various principles which are commonly inspired by the progress made in the 1970'ies with silicon-based technology for microelectronics. Examples of microfluidic applications are DNA-analyses involving principles such as the polymerase chain reaction for e.g. detection of single-nucleotide polymorphisms or assays for proteins using, e.g. capillary electrophoresis.

‘True’ microfluidic devices (e.g. with fluidic channels in the order of 100 μm diameter or less) do however suffer from a number of drawbacks, some of which are particularly announced for cell culturing devices designed for perfusion-type operation. As seen from the Hagen-Poiseuille equation the pressure drop in an e.g. 100 μm-channel with a flow becomes very large, putting high demands to a pump intended for operating at this scale, since such a pump must be able to precisely dispense very small volumes against a considerable back pressure. For this reason flows are often generated at this scale using so-called electroosmotic flow where a flow is created in a saline solution by exposing it to a large electrical potential. Such electroosmotic flow is however ill-suited for systems involving live (mammalian) cells.

Another problem encountered in microfluidics is one related to the ‘connection to the outside world’. Most equipment employed in biological labs, such as pumps and analytical equipment, is so much larger than microfluidic equipment that integration between the two scales becomes problematic. Connection points for a tube as small as 250 μm-diameter (as is readily available) to a chip are difficult to handle for the lab worker, and moreover may quickly introduce dead volumes several times the size of the volume of the microfluidic system. This problem is especially important for perfusion-type cell culture devices where the operational complexity and the long residence times of fluids in tubes connected to a microfluidic system increases the risk of upstream contamination. In the case of culture of mammalian embryos the culture time can amount to five days or more.

When operating with perfusion based cell culture systems it is of interest to be able to quickly analyse the growth conditions existing in a culturing chamber, so that results from the analyses may be used to modify the conditions in a feedback mechanism. For example, when a parameter value, such as pH or temperature or the presence or absence of a chemical entity, approaches a defined limit it may be necessary to change the conditions to bring the parameter value away from the limit value or to otherwise adjust the conditions to match the developmental state of the cell. In order to provide for such a feedback mechanism, a microbioreactor may be equipped with integrated sensors. Several uses or examples of integrated sensors have been described in the literature.

Thus, for example WO07/044699 describes a microbioreactor for setting up parallel studies of especially microbial strains of commercially relevant production organisms within the area of metabolic engineering studies. Integrated sensors in the growth chambers of the bioreactors of WO07/044699 allow signals from the sensors to be used in a feedback control set-up for adjusting the environmental conditions, typically pH, within the growth chamber by injecting liquid from reservoirs.

The principles underlying the microbioreactors of WO07/044699 make them suited for performing fed-batch type fermentations of cells by pulsing liquids into the growth chamber to maintain constant environmental conditions. However, the pulsating supply of liquid and the limited size of the reservoirs (i.e. 15-25 μL) relative to the size of the growth chamber make the systems ill-suited for long term perfusion-like operation, such as chemostat culture or continuous supply of medium to cells in the chamber (i.e. with a reservoir size of 25 μL and a pulse size of ˜270 nL fewer than 100 pulses are available).

Petronis et al. (2006) (BioTechniques 40:368-376) describe a microfluidic bioreactor device for long term culture of HeLa cells. This device is constructed from thermoplastic polymeric materials and contains an integrated indium tin oxide film to heat the growth chamber. The chamber further contains a temperature sensor, and a set-up with a computer allows cooperation between sensor and heat element so that the temperature of the chamber may be controlled in a feed-back fashion. The small size of the bioreactor of Petronis et al. (2006) allows quick control of the temperature in the chamber.

The above examples all employ integrated sensors. Integrated sensors do, however, suffer from considerable drawbacks. Monitoring of cells in systems as described above is limited to the parameters defined by the integrated sensors, and such systems are not flexible. For example, an unexpected reaction from the cells may create a desire to analyse for an entity or a parameter, which was not foreseen when the bioreactor was manufactured and the sensors were integrated. Therefore a system is desirable in which any given parameter may be analysed using a sensor that is located externally relative to the bioreactor.

Moreover, integration of a sensor in a microbioreactor may make the manufacture of the microbioreactor unnecessarily expensive and complex. If a microbioreactor with an integrated sensor is intended to be reused for medical purposes it will likely be difficult to obtain approval from authorities, such as the FDA or CE, because of the risk of contamination between uses involving different patients. It is therefore of interest that a microbioreactor is disposable due to the risk of cross-contamination between experiments, if a microbioreactor were to be reused. The considerations regarding unit cost will be especially pronounced if the bioreactor is produced with many different types of sensors to make the system more flexible, which types of sensors may not all be needed for a specific type of cell culture. Furthermore, disposable bioreactors are of particular interest for culturing cells that are to be reinjected into a patient, such as cells grown for regenerative medicine, immune therapy and IVF-purposes.

WO07/044938 in contrast describes a microfluidic sampling system for precisely withdrawing one or more micro- or nano-liter volumes of a sample. WO07/044938 describes a system created in elastomeric poly(dimethyl-siloxane) (PDMS), which system comprises an inlet port connected to a plurality of switches wherein the switches can direct a volumetrically metered sample of fluid via a channel to a sample well. The switches may be operably linked to one of the switches by channels providing for fluidic flow to move a fluid sample, wherein the volumetric metering loop can purge sample fluid from the system. The system also comprises a plurality of output ports operably linked to the sample wells by channels providing for fluidic flow to move the fluid sample. Thus, in other words WO07/044938 describes a microfluidic device for collecting metered volumes of samples in a number of distinct sample wells from a fluid flow. The device of WO07/044938 is however disadvantageously complex, both when seen from a manufacturing perspective and also during operation. The design of the device introduces potential dead volumes in the system, which makes it necessary to purge the channels of the device during collection of samples. These principles may introduce a delay if the analysis results obtained from the collected samples are to be used for feedback control.

It is therefore an aim of the present invention to provide a simple and inexpensive cell culturing system for culturing a biological cell, which cell culturing system comprises a culturing chamber that flexibly allows analysis of the contents of the culturing medium during culture of cells, especially downstream from the culturing chamber to minimise the risk of contamination of the culturing chamber. It is a further aim that such a device is suited for perfusion type operation on a scale and time appropriate for mammalian embryos. It is also an aim of the invention that such a device is disposable without incurring an excessive strain on the environment during its manufacture.

### DESCRIPTION OF THE INVENTION

The present invention relates to a cell culturing system comprising a culturing chamber for culturing a biological cell in a growth medium and a sensor for measuring a signal in the spent growth medium, wherein the culturing chamber is provided in a mesoscale bioreactor platform with an inlet opening for an influent steam of growth medium and a outlet opening for an effluent stream of spent growth medium, said spent growth medium being in fluid communication with a sample port for releasable adoption of the sensor. The outlet opening may be in fluid communication with an effluent channel provided in the platform, so that the sensor is adoptable in the sample port having fluid communication with the spent growth medium of the effluent channel. By positioning a sample port downstream from the culturing chamber it is possible to analyse a liquid stream from the culturing chamber with a sensor in the sample port. This set-up is particularly suited for a culturing chamber being perfused with a liquid, e.g. a growth or culturing medium. In this instance the culturing chamber has an inlet opening for an influent liquid and an outlet opening for an effluent liquid so that the culturing chamber may be perfused with liquid. However, the sample port may also be used in conjunction with a batch operated culturing chamber. In both operating principles the positioning of the sample port downstream from the culturing chamber will minimise the risk that the culturing chamber is contaminated with germs or pathogens, since the liquid stream will force germs away from the culturing chamber.

Spent medium will be led from the culturing chamber via the channel to the sample port where the liquid may be analysed with a sensor. Results from the analysis may then be used to modify the medium composition supplied to the cells in a feedback mechanism. From the sample port the liquid flow is led to a waste container, which may be located externally to the bioreactor platform. In one embodiment all chambers in the bioreactor platform, reservoirs, culturing chamber, sample port container and waste container take the form of upwards open wells. One or more of these chambers may further comprise a layer of a water-immiscible liquid.

The sensor is releasably adoptable in the sample port. This provides flexibility for analysing an effluent liquid from the culturing chamber, since any available sensor may be employed in the analysis. Likewise, the analysis of the effluent liquid is not limited to a single sensor, as a sensor in contact with liquid in the sample port may readily be replaced with another sensor. The sensor may be any available sensor that may provide an analysis of a relevant parameter for the culturing of a biological cell. Parameters of relevance to most cells, such as mammalian, bacterial, fungal, insect or plant cells, are pH, conductivity, dissolved oxygen (O₂), carbon dioxide (CO₂), glucose, flow velocity, temperature, and optical density. More specific parameters are individual nutrients, vitamins, signal molecules, hormones, metabolites, proteins or enzymes. Nucleotides, such as DNA's or RNA's, may also be relevant. The sensor may function on the basis of any type of signal. For example, the sensor may be capable of recording an electrical signal, an optical signal, a fluorescent signal, or the like, and the entity of interest may be measured directly or indirectly.

The sample port is constructed so that it provides physical access to a stream of liquid from the culturing chamber. The physical access to the stream of liquid from the culturing chamber allows that the liquid may be contacted with the sensor, i.e. the sensor is adoptable in the sample port, in order to analyse or measure the liquid with the sensor. Thus, in contrast to bioreactor systems with integrated sensors the sample port allows the liquid to be analysed with any available sensor. Thereby the sensor may be reused so that it is possible to construct an inexpensive and disposable bioreactor platform. Alternatively, it is also possible to withdraw a sample via the sample port for external analysis.

The sample port of the cell culturing system may comprise a container having a first opening for the effluent channel from the culturing chamber and a second opening for a waste channel for discharging the spent growth medium. Thus, the amount of liquid can be limited so that only an amount of liquid necessary for a given sensor is retained in the container. Moreover, the analysis performed with a sensor may therefore also provide a good reflection of current conditions in the culturing chamber due to it that spent medium may be removed from the sample port after analysis.

A sample port container may also comprise a third opening for a further inlet channel allowing introduction into the sample port of a liquid different from the effluent stream from the culturing chamber. Such a further inlet may be employed to dilute the effluent stream from the culturing chamber in order to bring an analyte concentration in the effluent stream within the detection range of a given sensor, or the volume of the effluent stream may be increased by adding the different liquid. A further inlet will also allow in situ washing or calibration of a sensor in the sample port.

In one embodiment the sample port is integrated on the mesoscale bioreactor platform. With a cell culturing system according to this embodiment, it is furthermore possible to conduct an analysis of the liquid stream nearly immediately after the liquid exits the culturing chamber. Due to the short delay between the culturing chamber and the sample port, this allows fast analysis results to be obtained, for example for cells with a quick metabolism where it may be relevant to quickly change culturing conditions.

In another embodiment, the cell culturing system according to the invention comprises two or more chambers for culturing a biological cell, wherein each chamber is in discrete fluid communication with the sample port. In this embodiment a single sensor may therefore be employed to analyse the effluent streams from each of the culturing chambers. The sample port, e.g. a sample port having a sample port container, is positioned to receive effluent streams from the two or more cell culturing chambers, wherein each cell culturing chamber has an outlet opening for an effluent stream of spent growth medium, which outlet openings are in fluid communication with the sample port. The outlet openings of the two or more cell culturing chambers may each be in fluid communication with an effluent channel providing fluid communication of the sample port with the spent growth media of the effluent channels. In this embodiment it is preferred that the sample port has an opening for a further inlet channel allowing introduction into the sample port of a liquid different from the effluent streams from the culturing chambers, as outlined above. Thus, the discrete effluent streams from the two or more cell culturing chambers may be analysed using a single sensor adoptable in the sample port; when a further inlet channel of the sample port is present it is possible to wash or calibrate the sensor between analysis of the effluent streams from the cell culturing chambers. For example, an effluent stream from a first cell culturing chamber may be analysed in the sample port using the sensor before washing the sensor, e.g. with water or buffer, while still located in the sample port and then analysing an effluent stream from a second cell culturing chamber.

The two or more culturing chambers may be provided in the same mesoscale bioreactor platform, or the chambers may be provided in separate bioreactor platforms. Likewise, the culturing chambers may share the same supply of growth medium/media, e.g. be in fluid communication with the same reservoir(s) for growth media, or each culturing chamber may have a separate supply of growth medium/media. For this embodiment it is relevant that the fluid communication between the respective culturing chambers and the sample port is discrete meaning that the effluent stream from one culturing chamber may be analysed independently from the effluent stream from another culturing chamber in the cell culturing system.

In one embodiment the sample port has an open, e.g. upwards open, container which may further comprise a layer of a water-immiscible liquid, such as an oil. A layer of a water-immiscible liquid will minimise the risk of contamination of the liquid, and moreover such a layer may also limit evaporation of solvent from the liquid, thereby helping to maintain the composition of the liquid, which is to be analysed in the sample port.

The sample port may also comprise a closable member or an elastic membrane. A closable member or an elastic membrane provides additional protection against particulate contaminants, such as germs or pathogens, while still providing physical access to the sample port. A closable member may take the form of a hinged lid or a sliding lid, and thus the liquid in the container of the sample port may be accessed by opening the lid. An elastic membrane is preferably made from a material having self-sealing capability. Thereby the liquid may be accessed by penetrating the membrane with an appropriately equipped sensor, e.g. with a needle or the like, so that upon removal of the sensor the self-sealing capability of the membrane will take effect.

The cell culturing system according to the invention may have an effluent channel which at one end is connected to the outlet opening of the culturing chamber and at the other end is connected to a coupling means and the sample port is connected to a hose having at the distal end complementary coupling means ensuring transport of spent medium from the effluent channel to the sample port. This principle of fluid communication between the culturing chamber and the sample port is especially advantageous when the sample port is external to the mesoscale bioreactor platform. This principle allows a quick coupling between the mesoscale bioreactor platform with the culturing chamber and the sample port due to the complementary coupling means. For example, the mesoscale bioreactor platform may be comprised in a cartridge that fits in the cell culturing system, so that insertion of the cartridge in the system will provide a coupling between the complementary coupling means, thus ensuring transport of spent medium from the effluent channel to the sample port. The coupling means may take the form of a male-female-type coupling, e.g. a luer-lock coupling, between the effluent channel and the hose, wherein the hose e.g. fits into the end of the channel. The coupling means may also comprise an upwards open well, so that the effluent channel provides fluid communication between the outlet opening of the culturing chamber and this upwards open well. The complementary coupling means may then comprise the hose, e.g. in the form of a tube, such as a teflon tube with an internal diameter of 0.5 mm or 0.25 mm, which is inserted into the upwards open well. Aspiration of liquid from the upwards open well into the teflon tube will then ensure transport of spent medium from the effluent channel to the sample port. A sample port located externally to the bioreactor platform may furthermore comprise means to control the temperature, such as a peltier element for heating or cooling, a heating coil, a heat exchanger employing liquids or gases etc. The temperature controlling means of the sample port may preferably control the temperature of the sample port independently of the temperature of the cell culturing chamber.

Furthermore, according to another embodiment of the invention a sterile filter is present in the stream of spent medium between the effluent channel of the culturing chamber and the sample port. A sterile filter may comprise a filter with a pore size of about 0.1 μm to about 0.5 μm, such as 0.22 μm or 0.45 μm. The sterile filter may be comprised in either of the coupling means discussed above, or it may be integrated into or otherwise part of the hose. The position of the sterile filter is not important, as long as substantially all spent medium from the culturing chamber to the sample port passes through the filter.

The mesoscale bioreactor platform of the cell culturing system may further comprise one or more medium reservoir chambers in fluid communication with the culturing chamber. Medium reservoirs comprised in the mesoscale bioreactor platform may minimise the risk of contamination of the culturing chamber compared to a mesoscale bioreactor platform with a culturing chamber being supplied with media from external reservoirs. When the mesoscale bioreactor platform comprises medium reservoirs, the cell culturing system may also comprise means to provide a flow from the reservoir(s) to the culturing chamber. Such means may take the form of an appropriate pump, which may be integrated into the mesoscale bioreactor platform, or external from the mesoscale bioreactor platform but integrated into the cell culturing system. In another embodiment the mesoscale bioreactor platform comprises two or more reservoirs for media with the reservoirs being in fluid communication with the culturing chamber via distinct conduits. The distinct conduits allow that the culturing chamber is supplied with medium from either one or a combination of the two or more reservoirs. This bioreactor platform is especially suited for culturing cells under perfusion conditions. Since the bioreactor platform has multiple reservoirs it is possible to adjust the composition of media supplied to the cell according to the current conditions of the cell.

The bioreactor platform is preferably constructed from one or more thermoplastic polymers, such as poly(methyl methacrylate) (PMMA), cyclic olefin copolymer or polystyrene (PS), although other materials such as metals, glasses or ceramics may also be used.

The flow of liquid into and out of the sample port may be controlled by providing a liquid driving force to the effluent channel. This liquid driving force may be provided from a pump, or alternatively from a siphoning effect created by differences in upper surface levels relative to a horizontal plane in case several upwards open chambers are included in the cell culturing system.

The cell culturing system according to the invention may be contained in an enclosure ensuring homeostasis. This enclosure may comprise a compartment with a gas supply for creating a laminar air flow around the mesoscale bioreactor platform, such as a laminar air flow positioned vertically around the mesoscale bioreactor platform with an air inlet in a bottom section of the compartment and an air outlet at a top section. When the cell culturing system comprises separate bioreactor platforms, the system may have a compartment for each bioreactor platform. The laminar air flow may also be horizontally oriented around the mesoscale bioreactor platform. When the mesoscale bioreactor platform comprises a culturing chamber and optionally one or more reservoir chambers which are upwards open, the gas composition of the laminar air flow may further be controlled in order to regulate diffusion of gases, such as CO₂ or O₂, into the chambers. The enclosure may also comprise a temperature regulation system, such as a metal block with a temperature regulation element.

The cell culturing system may also comprise a data processing unit capable of analysing a signal from the sensor and converting the signal to an analysis result. The system may also be capable of presenting the analysis result, e.g. on a display, to an operator who may use the result to modify operational parameters for the mesoscale bioreactor platform, such as in a feedback mechanism.

The cell culturing system may further comprise means to control operational parameters in the culture chamber, wherein the data processing unit is capable of sending commands to a control unit capable of regulating the means to control operational parameters. The operational parameters may comprise the chemical composition of the media supplied to the culturing chamber, the flow-rate of the liquid in the culturing chamber, the temperature of the bioreactor platform, the pH in the culturing chamber, the composition of gas surrounding the bioreactor platform etc. The operational parameters will be controlled according to the nature of each parameter. For example, flow-rates may be controlled using integrated or external pumps, the temperature may be controlled using e.g. a heating block, medium compositions may be modified by changing the flow-ratios between multiple reservoir chambers comprising different media, the pH of the culturing chamber may be adjusted by changing the gas composition, e.g. content of CO₂, above an upwards open culturing chamber. The system may also be capable of automatically employing signals from a sensor in the feedback mechanism.

In another aspect the invention relates to a method of measuring an effluent stream of spent growth medium from a culturing chamber in a cell culturing system according to the invention comprising the steps of: providing a biological cell in the culturing chamber, perfusing the culturing chamber with a growth medium entering through the inlet opening of the culturing chamber and exiting through the outlet opening, conveying the spent growth medium to a sample port, contacting the spent growth medium of the effluent channel with the sensor, and measuring a signal in the spent growth medium. The method may additionally comprise the step of adjusting the conditions in the culturing chamber on the basis of the measured signal.

The signal from the sensor or the analysis result obtained may be used by an operator or the control unit to evaluate the conditions of the cells, and on the basis of the signal or the analysis result as well as the conditions of the cells the operator may select appropriate modifications to the conditions in the culturing chamber. For example, the temperature may adjusted to maintain a constant temperature, or reach a higher or lower temperature. Likewise, the chemical composition or the pH may be changed or steps may be taken to retain these parameters at constant values.

Thus, the signals obtained from the sensor are employed to modify the medium composition supplied to cells in the culturing chamber in a feedback mechanism. A feedback mechanism may be employed to maintain steady state conditions for the cells, or the conditions of the cells may be adjusted to induce a change in the cells. For example, the concentration of a component in the liquid from the culturing chamber may be monitored, so that if the concentration of the component approaches a predetermined limit value, the composition of the medium supplied to the cells may be modified to maintain the concentration within the desired range. Likewise, the occurrence and detection of a specific component may lead to an adjustment of the composition of the supplied medium in order to induce a change in the cells.

### BRIEF DESCRIPTION OF THE FIGURES

The invention will now be described in more detail with reference to the following Figures, in which:

FIG. 1 schematically illustrates a sideview of the cell culturing system according to the invention.

FIG. 2 schematically illustrates a sideview of the cell culturing system according another embodiment of the invention.

FIG. 3 schematically illustrates a sideview of the cell culturing system according another embodiment of the invention.

FIG. 4 schematically illustrates a sideview of the cell culturing system according another embodiment of the invention.

FIG. 5 shows the layout of chambers and channels seen from above according to one embodiment of the invention.

FIG. 6 schematically illustrates the cell culturing system with a data processing unit and a control unit.

FIG. 7a shows a perspective drawing of a mesoscale bioreactor platform of the invention.

FIG. 7b shows a photo of a cell culture system with a mesoscale bioreactor platform of the invention.

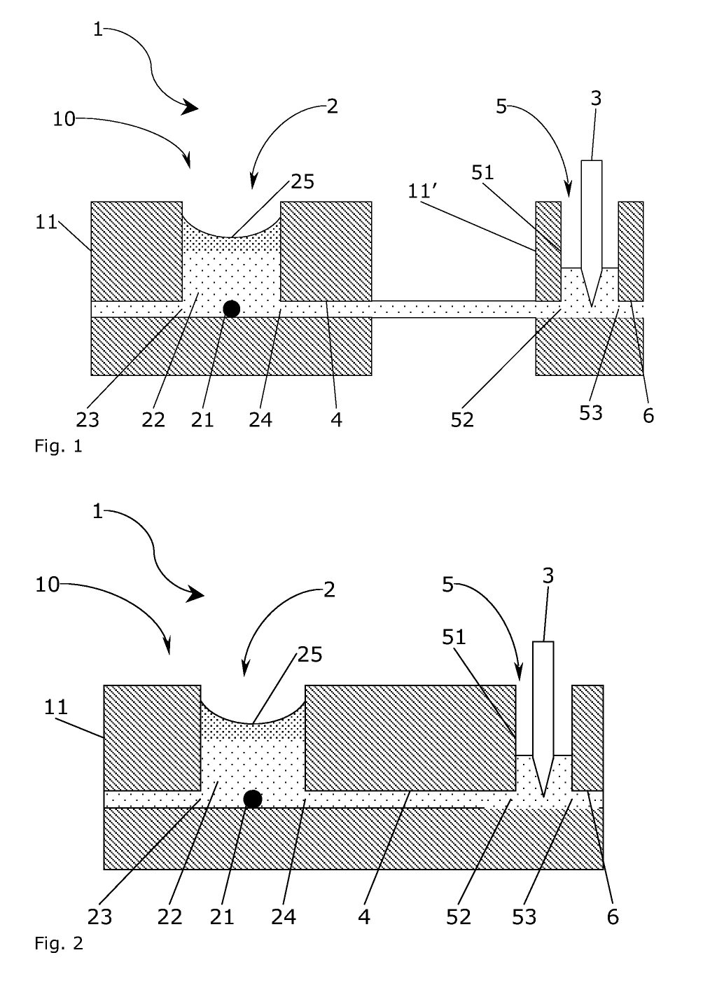

### DETAILED DESCRIPTION OF THE INVENTION

The present invention relates to a cell culturing system for culturing a biological cell in a growth medium and a sensor for measuring a signal in a sample port, as well as a method of analysing an effluent stream from a culturing chamber.

The term “bioreactor” of the present invention covers systems and devices suited for culturing biological cells. The disclosed bioreactors are especially suited for mammalian cells. In a preferred embodiment the mammalian cells are cells related to in vitro fertilisation, and the cells will comprise spermatozoa, oocytes, and/or embryos. However, as will be obvious to those skilled in the art the bioreactor platform may also be useful for other mammalian cell types, such as stem cells or cells of the immune system, such as monocytes, dendritic cells, T-cells and the like. In a preferred embodiment the mammalian cells are human cells. Furthermore, a mesoscale bioreactor platform as disclosed in the present invention may also be of utility in the culturing of cell types other than mammalian cells. For example, bacterial, yeast, fungal, plant, or insect cells may also be cultured in the bioreactor platform disclosed herein.

Bioreactors in the sense of the present invention will comprise a culturing chamber in fluid communication with the sample port. Bioreactors may also comprise reservoir chambers for media. In the context of the present invention the term “medium” refers to any liquid, which may be supplied to a cell being cultured in a culturing chamber in order to control the conditions of the cell. Thus, “medium” may refer to growth media containing salts, buffer components, nutrients, factors to induce an effect in the cells, such as differentiation, etc., or simply to a buffer without nutrients or the like. In general it can be said, that the medium refers to a liquid in which a cell has not been cultured.

In the context of this invention the term “mesoscale” is intended to cover a range of sizes where the smallest dimension of channels is in the range from around 100 μm to around 3 mm, although the channels may also contain constrictions. Likewise the culturing chamber may be of a depth of around 500 μm to around 5 mm or more, and the largest horizontal dimension may be from around 1 mm to around 50 mm. The size of any reservoir chambers should be sufficient to culture cells under perfusion conditions. It can generally be said that fluids in mesoscale fluidic systems will be flowing under laminar conditions, and fluidic systems with channels or chambers different from those defined above may well be described as “mesoscale” as long as fluids contained in the systems flow under laminar conditions.

The sample port, and its optional container, of the mesoscale bioreactor platform according to the present invention will be sufficiently sized to allow a sensor to contact the liquid in the container. In one embodiment the sample port is upwards open, and the sensor is brought into contact with the liquid simply by inserting it in the liquid. In addition to being sized to match the size of a sensor, the container may also be sized to obtain a specific linear flow-velocity through the container. For example, it may be desired to increase the cross-sectional area of the container relative to the effluent channel in fluid communication with the culturing chamber in order to decrease the linear velocity of the liquid flowing through the container. Thus, the residence time of the liquid flowing through the container may be modified in order to match the requirement, if any, of a sensor.

The bioreactor platform of the present invention is suited for operation under perfusion conditions. In this context the terms “perfusion” or “perfuse” mean that a generally continuous flow is applied to the culturing chamber of the device. This continuous flow is not limited to a certain flow-rate, but during the course of an experiment with a bioreactor platform of the present invention several different flow-rates may be employed. Suitable flow-rates are from around 1 μL/h to around 200 μL/min or more, although even lower flow-rates may also be used. Typical flow-rates are about 5, 7.5, 10, 12.5, 15 or 20 μL/h. The flow may be generated in pulses; at a low number of pulses at a small volume per pulse, such as 1, 2, 3 or up to 10 pulses of e.g. 0.5 μL, 1 μL etc., per time interval, such as per minute or per hour, e.g. 1 pulse of 1 μL per hour, the flow will in practice perform as a continuous flow. It should be emphasised that the flow may also be stopped if necessary, e.g. for performing various operations involving the contents of the culturing chamber. Furthermore, intermediate operation allowing rest to the biological cells is also contemplated.

The sample port is constructed in a way to allow physical access to a liquid from the culturing chamber in the container of the sample port. In this context the term “physical access” means that a sensor may be inserted into the liquid in the container to contact the sensor with the liquid. The culturing chamber may also allow physical access using an appropriate tool to insert or remove one or more cells from the culturing chamber, or to otherwise manipulate cells already present in the culturing chamber. When a sensor is designed so as to allow the sensor to contact the liquid in the sample port by being inserted into an upwards open chamber, by penetrating an elastic membrane, by being inserted after opening a lid etc., as is appropriate for a given sensor, the sensor can be said to be “adoptable” in the sample port. Furthermore, the sensor may also be removed from the sample port, and therefore the sample port can be said to be for “releasable adoption” of the sensor.

In one aspect, the present invention contains a “data processing unit”. With this term is meant a computer or similar device, which is capable of collecting a signal from a sensor in the system and converting it to data understandable to the operator, i.e. an analysis result. The data processing unit may also comprise a display or similar to present this analysis result. In general, the sensor can read an observation, such as an optical signal, e.g. light intensity, or an electrical signal based on e.g. a reference electrode, and convert it to an electrical signal. When the data processing unit receives this electrical signal it may be compared to a standard corresponding to a known parameter value and converted to an analysis result in the appropriate units to be presented as an analysis result.

The data processing unit may also be capable of sending commands to a “control unit” for controlling operational parameters in the mesoscale bioreactor platform. The commands may be based on the signal from the sensor or the analysis result, and will be send to the control unit as an electrical signal. The signal from the sensor may be compared to an instruction set containing a list of commands to be send to the control unit in response to a given parameter value. For example, an increase in pH may result in a command to increase the concentration of CO₂ above an upwards open culturing chamber, thus increasing the concentration of CO₂ of the liquid and thereby decreasing the pH in the culturing chamber. A suitable pH for IVF purposes is between 7.2 and 7.5, in particular an optimum pH is in the range of 7.25 to 7.45. The commands may also be given to the control unit by an operator in order to change the conditions in the culturing chamber.

The cell culturing system 1 of the present invention comprises a culturing chamber 2 for culturing a biological cell 21 in a growth medium 22 and a sensor 3 for measuring a signal in the spent growth medium, wherein the culturing chamber 2 is provided in a mesoscale bioreactor platform 10 with an inlet opening 23 for an influent steam of growth medium and a outlet opening 24 for an effluent stream of spent growth medium, said spent growth medium being in fluid communication with a sample port 5 for releasable adoption of the sensor 3. In one embodiment the outlet opening 24 is in fluid communication with an effluent channel 4 provided in the platform 10, and the sensor 3 is adoptable in the sample port 5 having fluid communication with the spent growth medium of the effluent channel 4. The sample port 5 may further comprise a container 51 having a first opening 52 for the effluent channel 4 from the culturing chamber 2 and a second opening 53 for a waste channel 6 for discharging the spent growth medium.

The sample port container 51 may also comprise a third opening for a further inlet channel (not shown). This further inlet channel allows the introduction into the sample port 5 of a liquid different from the effluent stream from the culturing chamber 2, for example water, a buffer, a calibrating liquid, a liquid to clean the sensor, etc. Water, e.g. distilled water, demineralised water, milli-Q water, etc. may be introduced into the sample port container 51 to dilute the contents of the effluent stream from the culturing chamber 2; this step may be appropriate to bring the concentration of an analyte of interest into a concentration range appropriate for a sensor for the analyte. Water may also be added to increase the volume of the liquid in the sample port container 51. For example, water may be added to increase the volume so that it will become appropriate for analysing with a specific sensor, e.g. a pH electrode. In the former case it may be important to control the amount of water added in order to calculate the analyte concentration in the effluent stream from the culturing chamber. In the latter case it may also be of interest to add a specified amount of water, although in case the effluent stream from the culturing chamber comprises a buffer, moderate dilution of the effluent stream will not change the pH considerably. For example, a volume of flow may be collected for a time to reach a desired volume, such as about 15 μL, before adding water to adjust the volume to approximately 30-100 μL or more allowing the pH to be measured using a standard pH-electrode. Typical flow-rates may be 5, 7.5, 10, 12.5, 15 or 20 μL/h, and the time for collection may be calculated from the flow-rate. For analysis of some parameters, such as pH, the exact volume collected may not be important. However, the volume should be sufficient for analysis. Despite the addition of water the recorded pH-value will closely reflect the pH of the effluent stream from the culturing chamber due to the buffer present in the growth medium. A further inlet will also allow the sensor, e.g. a pH-electrode, to be washed or calibrated in situ without removal of the sensor from the sample port.

The mesoscale bioreactor platform 10 may be constructed from a substrate 11 into which the culturing chamber 2 and the channel 4 are defined. The substrate is preferably a thermoplastic polymer, such as poly(methyl methacrylate) (PMMA), cyclic olefin copolymer or polystyrene (PS), and in a preferred embodiment at least the bottom of the culturing chamber 2 and the optional reservoir chamber 8 are transparent to light within the visible range of the electromagnetic spectrum. In another preferred embodiment, the bottom materials are also transparent to light within the ultraviolet spectrum, such as between 250 and 400 nm. The substrate 11 may also comprise other materials, such as metals, glasses or ceramics, or the substrate 11 may contain a combination of several types of materials. For example, a polymeric material comprising structures to make up chambers and channels may be glued or otherwise attached to a glass slide, such as a microscope slide. Chambers and channels may also be defined in a polydimethylsiloxane (PDMS) substrate that may be attached to a slide of a more rigid material. For example, a PDMS-substrate with chambers and channels may be cast or moulded, and after curing one surface of the substrate may be treated with an oxygen plasma followed by attachment to a glass slide.

The sample port 5 may likewise be constructed from a substrate 11′ of the same general characteristics of the substrate 11. The container 51 be constructed as the culturing chamber 2 in substrate 11.

The container 51 of the sample port 5 may be open to the ambient surroundings, and in one embodiment the container 51 comprises an upwards open well. Such openness allows a sensor 3 to contact the liquid, such as an effluent stream of liquid from the culturing chamber 2, in the container 51 by inserting the sensor 3 into the liquid. It is generally necessary to ensure control of the flow of liquid entering the container 51 via the effluent channel 4 and also the flow of liquid leaving the container 51 via the waste channel 6. When substantially equal flow-rates exist in the two channels 4 and 6 a steady state with respect to the level of liquid in the container 51 will exist. The flow-rates in the channels 4 and 6 may be controlled using one or more pumps (not shown); the pumps, e.g. of a peristaltic type or a microannular gear pump, may be integrated in the bioreactor platform if the sample port 5 is also integrated, or the pumps may be located externally.

When the mesoscale bioreactor platform comprises a culturing chamber 2 being upwards open the medium in the culturing chamber 2 may be provided with a layer of a water-immiscible liquid 25, such as paraffin oil. This water-immiscible liquid 25 may then be said to form a closure on the culturing chamber 2 preventing evaporation of the solvent from the culturing chamber 2, and further allowing the pH to be controlled by adjusting the pressure of CO₂ above the culturing chamber 2.

Externally located pumps, such as a peristaltic pump, a piston pump, a syringe pump, a membrane pump, a diaphragm pump, a gear pump, a microannular gear pump, or any other appropriate type of pump may provide a positive relative pressure to the inlet opening 23, thus dispensing liquid from the culturing chamber 2 into effluent channel 4 and further into the sample port 5. In contrast a negative relative pressure may be applied to waste channel 6, thus aspirating liquid into the sample port 5 via effluent channel 4 from the culturing chamber 2. Such respective positive and negative relative pressure may also be provided in the cell culturing system 1 fitted with appropriate pumps.

If several upwards open chambers, e.g. a reservoir chamber 8 and a culturing chamber 2, and optionally a sample port 5 with a container 51, are contained in the same mesoscale bioreactor platform, the differences in the upper surface levels relative to a horizontal plane between the chambers will determine a pressure differences between the chambers. This pressure difference will provide that a liquid at a higher level is siphoned towards a chamber having a lower upper surface level. In the context of this invention, this principle of providing a liquid driving force is termed a “siphoning effect”.

When the mesoscale bioreactor platform 10 has a culturing chamber 2 with a higher surface level of liquid than that of a container 51 of a sample port 5 located downstream from the culturing chamber 2, which in turn is higher than the surface level of liquid in a waste container (not shown) located downstream of the sample port 5 integrated on or externally from the mesoscale bioreactor platform 10, a liquid driving force will be formed driving a liquid flow from the culturing chamber 5 to the container 51 and from there to the waste container while retaining the liquid level in the container 51 approximately constant. This principle may also be expanded by placing a reservoir chamber 8 upstream from the culturing chamber 2 with this reservoir chamber 8 having a higher level of liquid than the culturing chamber 52. The liquid driving force providing using this siphoning effect may also be supplemented with externally located or integrated pumps. For example, the air pressure above the reservoir 8 may be increased, or liquid may be aspirated from the container 51 or the waste container, resulting in a flow from the reservoir chamber 8 to the culturing chamber 2 further to the container 51 and into the waste container. This way it is possible to retain the liquid levels at steady states in both the culturing chamber 2 and the container 51.

In one embodiment (not shown) of the mesoscale bioreactor platform the sample port further comprises a closable member or an elastic membrane providing physical access to the container. This construction allows access to the container while maintaining a minimal risk of contamination. The closable member of the mesoscale bioreactor platform may have the form of a lid, which may be hinged or sliding, and the elastic membrane may have a self-sealing capability. Thus, the container of the sample port may be accessed with a sensor by opening the hinged or sliding lid and inserting the sensor into the liquid in the container. An elastic membrane, in particular one with self-sealing capability, may be penetrated with a sharp object to allow access of the sensor to the liquid in the container.

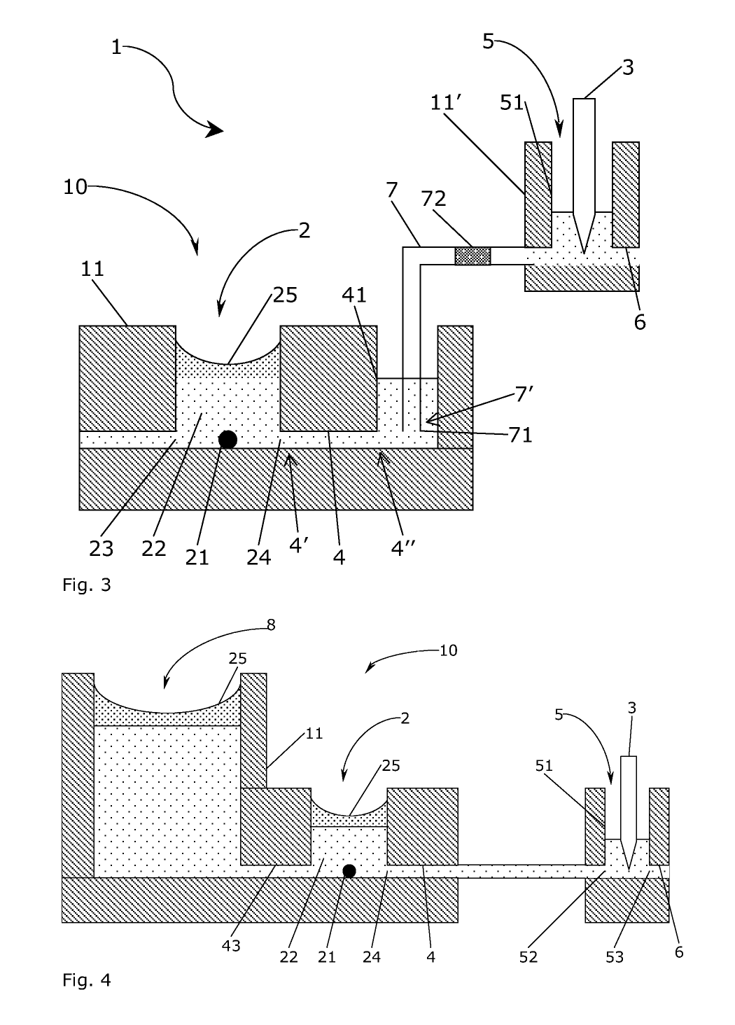

In the preferred embodiment illustrated in FIG. 3, the cell culturing system 1 has an effluent channel 4, which at one end 4′ is connected to the outlet opening 24 of the culturing chamber 2 and at the other end 4″ is connected to a coupling means 41 and the sample port 5 is connected to a hose 7 having at the distal end 7′ complementary coupling means 71 ensuring transport of spent medium from the effluent channel 4 to the sample port 5. FIG. 3 shows the coupling means 41 in the form of an upwards open well into which the distal end 7′ of the hose 7 is inserted, so that the distal end 7′ represents the complementary coupling means 71. The hose is preferably a teflon tube of 0.5 mm or 0.25 mm inner diameter. Several types of attachment systems are readily available, such as those supplied by Mikrolab Aarhus A/S (Aarhus, Denmark), for attaching the hose 7 to the sample port 5. The sample port 5 is preferably external to the mesoscale bioreactor platform 10. This allows that the mesoscale bioreactor platform 10 containing the culturing chamber 2 is constructed independently from the sensor 3, so that an inexpensive mesoscale bioreactor platform 10 can be manufactured, used and disposed of after use, while the possibly expensive sensor 3 in the sample port 5 may be used multiple times. In the embodiment of FIG. 3 it is moreover difficult for cells 21 or the like from the culturing chamber 2 to reach the sensor 3 in the sample port 5. However, a sterile filter 72 may preferably also be present in the stream of spent medium between the effluent channel 4 of the culturing chamber 2 and the sample port 5. Such a filter 72 may have a pore size of about 0.1 μm to about 0.5 μm, such as 0.22 μm or 0.45 μm. Filters with these characteristics are likewise readily available commercially.

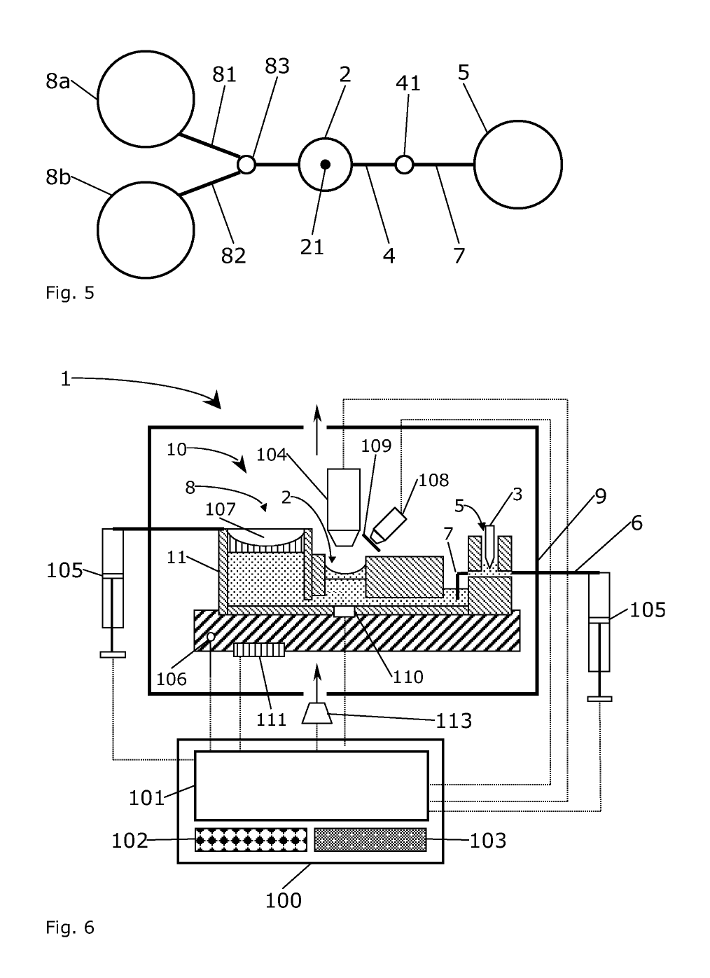

In another preferred embodiment of the invention as illustrated schematically in FIG. 5, the mesoscale bioreactor platform 10 is provided with two reservoir chambers 8a,b, respectively, for growth media with the reservoir chambers 8a,b being in fluid communication with the culturing chamber 2 for a biological cell 21 via distinct channels 81, 82, respectively. The distinct channels 81 and 82 may be in fluid communication with the culturing chamber 2 via a manifold 83, or they may connect directly to the culturing chamber 2 (not shown). The reservoir chambers 8a,b and the culturing chamber 2 are preferably upwards open. When these are upwards open, the aqueous liquids therein may each be provided with a layer of a water-immiscible liquid, such as paraffin oil. This water-immiscible liquid may be said to form a closure on the upwards open chambers 2, 8a,b. Such a closure will prevent contamination with particles, such as germs or pathogens, prevent evaporation of solvents from the chambers, and provide a heat insulating layer. Importantly, a layer of a water-immiscible liquid will allow diffusion of gases, such as CO₂ or O₂, into or out of the aqueous liquid in the chamber. Control of the CO₂ above a chamber may be used to control the pH of the aqueous liquid in the chamber, since a high CO₂ pressure will decrease the pH in the liquid, whereas a low CO₂ pressure may allow the pH to increase.

The culturing chamber 2 is in fluid communication via effluent channel 4 with coupling means 41, which is likewise located on the mesoscale bioreactor platform 10. Coupling means 41 connect to complementary coupling means on the hose 7. The hose 7 provides a flow of liquid, i.e. spent medium, from the culturing chamber 2 to the sample port 5.

The mesoscale bioreactor platform of the invention is not limited to a single culturing chamber. In some embodiments the mesoscale bioreactor platform comprises several culturing chambers; when multiple culturing chambers are present they may be arranged in one or more groups. The chambers in one group may be serially connected with channels for liquid streams, and the groups may be connected in parallel with channels for liquid streams. When the bioreactor platform is designed to employ the siphoning effect as discussed above, the bioreactor platform may also comprise multiple culturing chambers. The mesoscale bioreactor platform may employ a single sample port so that all fluid streams from the culturing chambers are led to the same sample port, or each group of serially connected culturing chambers may have a sample port. When multiple culturing chambers are connected to the same sample port, the liquid being analysed can be said to represent an average value for the culturing chambers.

In a specific embodiment, the cell culturing system comprises two or more chambers for culturing a biological cell, wherein each chamber is in discrete fluid communication with the sample port. The discrete fluid communication between each chamber and the sample port allows that the effluent streams of the chambers may be analysed separately. In this context, the two or more chambers may also mean two or more groups of culturing chambers where for each group an effluent channel is in discrete fluid communication with the sample port. For example, the cell culturing system may comprise e.g. from 2 to 10, such as 6, bioreactor platforms with each bioreactor platform comprising reservoirs, e.g. 1 reservoir or 2 reservoirs, for growth media in fluid communication with a cell culturing chamber or a group of e.g. 4 to 20, such as 12, serially connected cell culturing chambers. When a bioreactor platform comprises a group of serially connected cell culturing chambers, each group will typically be used for cells of the same origin. Thus for example, for IVF purposes a group of culturing chambers may contain oocytes from the same patient. The serially connected culturing chambers of a bioreactor platform will be in fluid communication with a coupling means allowing connection to a complementary coupling means and providing a flow of effluent stream from the culturing chamber or group of culturing chambers to the sample port; the sample port is preferably located externally to the mesoscale bioreactor platforms. All variations described above for other embodiments of the invention may apply equally to this embodiment, although it is preferred that the sample port has an inlet channel for a liquid different from the effluent streams from the culturing chambers. Separate bioreactor platforms allow that cells, such as embryos or stem cells etc., from different individuals are cultured separately so that contact between cells from different individuals is prevented. For example, it may be prevented that signaling molecules, such as hormones, cytokins, chemokins or the like, secreted from a cell from one individual may effect cells from another.

Separate bioreactor platforms employed in the cell culturing system may further comprise means to identify the individual providing the cells cultured in the bioreactor platform. For example, each bioreactor platform may have a label or code, such as a colour code, a number, a pin code, an RFID chip or the like, allowing simple identification of the bioreactor platform and its contents.

When the cell culturing system comprises separate bioreactor platforms and these are in fluid communication with the sample port via a coupling-complementary coupling as described above it is possible to connect and disconnect a bioreactor platform from the sample port without affecting any other bioreactor platforms in the cell culturing system. Thus, it is possible to culture cells in separate bioreactor platforms independently from each other. For example, in a cell culturing system comprising e.g. six bioreactor platforms each of the six platforms may be inserted in the cell culturing system at any time and connected to the sample port via the coupling-complementary coupling. This allows that a cell culturing process, e.g. an IVF procedure, is initiated at a point of time independent from the time elapsed in IVF procedures performed in other bioreactor platforms in the cell culturing system.

A cell culturing system of the invention comprising multiple separate bioreactor platforms may analyse the effluent streams from the culturing chambers of each platform using only a single sample port. The flow from each bioreactor platform, i.e. the effluent stream from the culturing chamber(s), may be directed to the sample port when an analysis is desired. When an analysis has been performed for one effluent stream, the effluent stream from another bioreactor platform may be directed to the sample port for analysis. If the sample port comprises a further inlet channel for the introduction into the sample port of a liquid different from the effluent stream (as outlined above) it is further possible to wash the sensor in the sample port between analyses.

The chambers of the mesoscale bioreactor platform in the cell culturing system of the present invention are not limited to a particular shape. However, in a preferred embodiment the shape of the chambers may be generally described as cylindrical with an essentially round circumference. The diameter of this circumference may be larger or smaller than the height of the cylinder. The height of the cylinder will normally follow the vertical axis. In one embodiment the diameter of the cylindrical culture chamber may be from around 2 to around 6 mm, for example around 2.5 mm or 4 mm, and in another embodiment it may be from around 20 to around 30 mm, for example around 25 mm. The depth of these cylindrical culture chambers may be from around 0.5 to around 2 mm, for example around 1.5 mm. Reservoir chambers and waste chambers will generally have a greater depth than the culture chambers, typically around 6 mm. The reservoir chambers will generally be of a volume to sufficiently perfuse the culturing chamber with medium for the full duration of the culturing period. Thus, in one embodiment the volume of a reservoir chamber is at least 10 times the volume of a culturing chamber. In another embodiment the volume of a reservoir chamber is at least 100 times the volume of a culturing chamber

In other embodiments the culture chamber may be generally box-shaped. This box-shape may take the form of a generally flat box with rectangular sides, or the box may be closer to a cube in shape. In one embodiment the culture chamber may be of a width of around 5 to around 10 mm with a length of up to around 50 mm. The depth of such box-shaped culture chambers may be from around 0.5 to around 2 mm.

Channels and chambers of the mesoscale bioreactor platform of the present invention may be formed by joining a first substrate layer comprising structures corresponding to the channels and chambers with a second substrate layer. Thus, the channels are formed between the two substrates upon joining the substrates in layers, and chambers may correspond to the thickness of the layer. The mesoscale bioreactor platform is not limited to two substrate layers. In certain embodiments multiple substrates may be used where each of the substrates may comprise structures for channels and chambers as appropriate. These multiple substrates are then joined in layers so as to be assembled as a mesoscale bioreactor platform.

The structures corresponding to the channels and chambers in the substrates may be created using any appropriate method. In a preferred embodiment the substrate materials are thermoplastic polymers, and the appropriate methods comprise milling, micromilling, drilling, cutting, laser ablation, hot embossing, injection moulding and microinjection moulding. Injection moulding and microinjection moulding are preferred techniques. These and other techniques are well known within the art. The channels may also be created in other substrate materials using appropriate methods, such as casting, moulding, soft lithography etc.

The substrate materials may be joined using any appropriate method. In a preferred embodiment the substrate materials are thermoplastic polymers, and appropriate joining methods comprise gluing, solvent bonding, clamping, ultrasonic welding, and laser welding.

A preferred embodiment of the mesoscale bioreactor platform comprises two reservoir chambers 6 mm depth with respective diameters of 14 mm and 12 mm. Two effluent channels lead from each of the reservoir chambers to two separate manifolds. From each manifold a channel leads to the first of a series of six culture chambers, so that the two groups each of six culture chambers are connected in parallel with the reservoir chambers. Each culture chamber has a diameter of 2.5 mm and a depth of 1.5 mm. From each of the last culture chambers in the two series, a channel leads to another chamber of 7.9 mm diameter and 6 mm depth. This chamber functions as the coupling means into which a hose may be inserted in order to provide effluent liquid from the culture chambers to an off-platform sample port. The culture chambers are arranged in a 3×4 pattern confined within a 25 mm diameter circle. The substrate defines a wall of 4.5 mm height with an inner surface corresponding to the 25 mm circle. This inner surface further defines a well for a water-immiscible liquid, so that the culture chambers will share a single closure formed by the water-immiscible liquid.

The cell culturing system may be contained in an enclosure ensuring homeostasis, and the system may comprise a data processing unit capable of analysing a signal from the sensor and converting the signal to an analysis result. The cell culturing system may also comprise means to control operational parameters in the culture chamber, wherein the data processing unit is capable of sending commands to a control unit capable of regulating the means to control operational parameters. FIG. 6 schematically shows a cell culturing system 1 contained in an enclosure 9. The enclosure 9 may be an appropriately sized box made of a polymeric material or a metal.

The enclosure 9 may be supplied with gases to create a laminar air flow 113 around the bioreactor platform 10. This “laminar air flow” 113 describes a situation where air is flowing through the enclosure 9 in a pattern virtually free of turbulence, and these conditions may serve to lift air-borne particular material away from the chambers 2,8,5, especially from upwards open chambers, of the cell culturing system 1, thereby minimising contamination of the cells and media in the chambers. In one embodiment the laminar air flow 113 is supplied to the enclosure 9 from below the bioreactor platform 10 to one or more exits above the bioreactor platform 10 so that the air is moving in a generally upwards direction. In another embodiment, the laminar air flow is oriented along the surface of the bioreactor platform, i.e. in a substantially horizontal orientation. The laminar air flow 113 may be composed of atmospheric air, though in a preferred embodiment the content of CO₂ is increased relative to atmospheric air to e.g. approximately 2-10%, or more preferably 5%. In other embodiments the content of O₂ may also be increased or decreased. The pressure of the laminar air flow may be essentially identical to that of the ambient air. However, the pressure is preferably increased relative to the ambient air. When the content of CO₂ or O₂ or other gases of the laminar air 113 flow is increased, the flow may further be controlled so that the pH of liquids in the chambers 2,8,5 of the cell culture system 1 may be regulated. The linear flow velocity of the laminar air flow is typically in the range 50 μm/s to 0.1 m/s.

The data processing unit 100 of the system 1 may have a user interface 102 and a display 103 for presenting analysis results. The data processing unit 100 may also have a control unit 101 capable of regulating means for controlling operational parameters in the system. The control unit 101 may receive commands from the data processing unit 100, which commands may be entered by an operator into the user interface 102 allowing the operator to manually control the operational parameters, such as flow-rates in the channels and chambers, distribution of flow from different reservoirs, temperature, CO₂-pressure, the laminar air flow, pH etc. The control unit 101 may be set up to control the operational parameter values of the cell culturing system 1 in a fully automated, pre-determined sequence of events, or a fully automated sequence of events determined by signals from the sensor 3 of the system 1, a manually operated sequence of events, or any combination of these operating principles.

The cell culture system 1 may be provided with air tight connections to the substrate 11 of the bioreactor platform 1 so that separate compartments 107 in which the pressure may be controlled independently are formed. Such compartments 107 are preferably formed above the reservoir chambers and the waste container, which are upwards open. This allows that a positive relative pressure is applied to the reservoir chambers of the bioreactor platform 1 and/or a negative relative pressure to the waste container. The application of such pressures will create a flow of liquid from one or more of the reservoir chambers towards the culturing chamber, and from there to the sample port and then the waste container. The gas applied to the reservoirs with a positive relative pressure to the reservoir chambers may be of any composition. Thus, the gas may be air or it may be premixed with e.g. 2-10% CO₂, preferable 5% CO₂, and/or it may be a trigas with 2-20% O₂.

The system 1 may comprise one or more pumps 105, which may be in fluid communication with air inlets to a compartment 107 or the waste channel 6. The compartment 107 encloses a reservoir chamber 8, and thereby it is possible to increase the pressure above the liquid in the reservoir 8 and create a flow from the reservoir to the culturing chamber 2. The pump 105 in communication with waste channel 6 may create a negative relative pressure in this channel 6, which in return will create a flow of liquid into the sample port 5 from the culturing chamber 2. The system 1 may comprise a pump 105 for each reservoir chamber 8, in case several are present, or it may comprise a single pump 105, or the number of pumps 105 may fall between these two values. In case the system 1 has fewer pumps 105 than the number of reservoir chambers the system 1 may also comprise appropriate valves (not shown) enabling the composition of liquids supplied to the culturing chamber 2 of the mesoscale bioreactor platform 10 to be controlled with respect to the contents of the reservoirs. It is also possible to create a flow using only the pump 105 connected to the waste channel; in this case there must be an air inlet into the compartment 107 for the liquid to flow in the system 1. If more reservoir chambers are present, a flow from one reservoir may be blocked by blocking this air inlet. Pump 105 may be a piston pump, a syringe pump, a membrane pump, a diaphragm pump, or any other appropriate type of pump for pumping gases or liquids.

The cell culture system 1 may also be fitted with a system 111 to regulate the temperature of the mesoscale bioreactor platform 10. The temperature regulation system 111 may further comprise one or more temperature sensors 106 coupled with the data-processing unit 100 allowing the temperature to be controlled via the control unit 101. The temperature regulation system 111 may for example comprise a metal block, such as an aluminium block, shaped to house the bioreactor platform 10 and comprising a coil of an electrically conductive wire, a peltier element, tubes for a heating and/or cooling liquid, or similar. In a preferred embodiment the control unit is equipped with an aluminium block with a heating element 111 and a temperature sensor 106; this temperature sensor is connected to the data-processing unit 100. In another preferred embodiment the system 1 is equipped with a block of a transparent material, such as glass, containing the heating element 111. The data-processing unit 100 in this embodiment may use signals from the temperature sensor 106 in a so-called model predictive control (MPC) algorithm to precisely regulate the temperature of the mesoscale bioreactor platform 10 by controlling the power supplied to the heating element.

In addition to this temperature sensor 106 the system 1 may be equipped with optical detection and observation systems. Optical detection and observation systems are preferably located so as to observe the culturing chamber, but the container of the sample port may also be observed. These optical systems could comprise a light source 108, such as a light emitting diode (LED), a light bulb, a mercury lamp, a laser or the like, appropriate filters 109 and a photo detector 110. LED's may be of a type to emit white light or they may be of a type emitting light of a relatively narrow range of wavelengths. This latter type of LED's are appropriate for measuring optical densities of wavelengths corresponding to that characteristic for the LED or for exciting fluorescent entities to emit light of a characteristic wavelength which may then be detected as a fluorescent signal. Alternatively mercury lamps are also appropriate components for detection of fluorescent signals when coupled with suitable light filters 109 and photodetectors 110.

In a preferred embodiment the cell culture system 1 is equipped with a digital or an optical microscope 104 positioned so as to observe the culturing chamber 2. The display 103 of the data processing unit 100 may allow the culturing chamber 2 and its contents to be visualised and monitored via a transmission of a signal from the microscope 104. In yet another embodiment the system 1 further comprises visualisation software capable of monitoring any cells growing in the culturing chamber 2 and, depending on the morphology of the cells, sending commands to pumps 105 controlling the pressure applied to the reservoirs 8 and/or the waste channel 6 from the sample port 5, the gas supply generating a laminar air flow 113 and the heating/cooling system 111 to regulate the temperature of the mesoscale bioreactor platform 10. The morphology of the cells may involve the number, sizes, shapes or orientation of cells or a combination. The morphology may also involve fluorescent signals or colorimetric signals from the optical detection systems.

The data-processing unit 100 is capable of collecting signals from the sensor 3, as well as from the optional temperature sensor 106 and signals generated from the microscope 104 and the photodetector 110 and sending commands to the control unit 101 to control the operational parameters for the culturing chamber 2, such as to control the flow-rate of liquid streams, e.g. the ratio of flow from different reservoir chambers, to regulate the temperature and/or the laminar air flow as appropriate. Control of these operational parameters may be based on a predetermined chronological series of events, or the commands may be based on the signals measured by the sensor 3 in the liquid from the culturing chamber 2, the temperature sensor 106, and/or the photodetector 110 in a feedback-type loop. In case a predetermined sequence of commands is employed this could for example involve perfusing an embryo with growth medium from one reservoir for a set number of days at a given flow-rate before changing the perfusion to growth medium from another reservoir at the same or a different flow-rate for the remainder of the culturing period while at all times maintaining the temperature at 37° C.

The control of the operational parameters may also involve a more complex set of instructions employing signals from the sensor 3, the temperature sensor 106, and/or the photodetector 110. For example, when a signal from a sensor 3 or an observation indicates that an event has occurred in the culturing chamber the instruction set may contain an instruction for the control unit 101 to counter the event and maintain a stable operational parameter value for parameters such as pH, temperature, or nutrients perfused to the culturing chamber 2, or the instruction set may contain an instruction for the control unit 101 to apply a new set of conditions to the cells in the culturing chamber 2. This could for example be that when a certain morphology is observed for an embryo in the culturing chamber 2, the flow is changed from the growth medium contained in one reservoir to that of another reservoir. Thus, these conditions could involve parameters such as flow-rate, temperature, pH, distribution of flows from the different reservoirs, or the like.

An example of a set-up employing signals from the sensor 3, the temperature sensor 106, and/or the photodetector 110 to determine the operational parameters could be that if a temperature sensor 3 or 106 indicates that the temperature is outside a set interval, the control unit 101 will send a command to the temperature regulating system 111 to heat or cool the mesoscale bioreactor platform 10 so that the temperature will once again be brought within the set interval. Likewise, with an indication from a pH sensor 3 that the pH is moving away from a set range, the gas supply may for example be adjusted so that an increased amount of CO₂ is applied to the enclosure 9 containing the mesoscale bioreactor platform 10. Levels of O₂, glucose and other metabolites (e.g. pyruvate and lactate) and energy (ATP/ADP) may also be used for controlling the operational parameters.

Regardless of the principle of operation the data-processing unit 100 may create a temporal log of the signals collected from the sensor 3, the temperature sensor 106, and/or the photodetector 110 of the system 1. This temporal log may also contain information about events in e.g. the culturing chamber 2 of a mesoscale bioreactor platform 10 or commands employed to control operational parameters for the system 1. The temporal log may advantageously be coupled with the information from a RFID tag (not shown) on the mesoscale bioreactor platform 10, and in one embodiment the system 1 comprises a device (not shown) to read the RFID-tag. This way a temporal log may be easily linked with a data tag containing information about the origin of the cells in the culturing chamber 2, such as the name and identity of the person providing the cells, as well as the identity of the operator.

The cell culture system 1 may simply be designed to hold a single mesoscale bioreactor platform 10. However, in another embodiment the system 1 may contain e.g. up to six mesoscale bioreactor platforms 10 in one system 1.

In the cell culture system 1 of the invention, the bioreactor platform 10 is preferably designed in the form of a cartridge fitting in the system 1, where the system comprises the sample port 5. Insertion of the cartridge into an appropriately designed seat or similar in the system 1 will ensure that the bioreactor platform 10 is connected correctly, i.e. that air tight connections are made between the substrate 11 forming the compartments 107 with connections to the pump(s) 105. Likewise, the culturing chamber 2 will be positioned correctly relative to the optional microscope 104, and the sample port 5 will be aligned with the sensor 3 for bringing the sensor 3 in contact with liquid in the container 51 of the sample port 5.

Insertion of the cartridge containing the bioreactor platform 10 into its seat in the cell culture system 1 will further ensure efficient heat transfer between the temperature regulating system 111 and the bioreactor platform 10. When the cartridge is inserted in the system 1 any optical detection or monitoring systems being part of the system 1 will be appropriately aligned with culturing chamber 2 or channels in the mesoscale bioreactor platform 10.

Thus, the application of a mesoscale bioreactor platform 10 contained in a cartridge fitting into a suitably designed cell culture system 1 will allow a quick coupling between the mesoscale bioreactor platform 1 and the system 1. Correct insertion of the cartridge will preferably be obvious to the operator.

In another aspect the invention relates to a method of measuring an effluent stream of spent growth medium from a culturing chamber in a cell culturing system according to the invention, comprising the steps of: providing a biological cell in the culturing chamber, perfusing the culturing chamber with a growth medium entering through the inlet opening of the culturing chamber and exiting through the outlet opening, conveying the spent growth medium to a sample port, contacting the spent growth medium of the effluent channel with the sensor, and measuring a signal in the spent growth medium. The method may further comprise the step of adjusting the conditions in the culturing chamber on the basis of the measured signal.

In the cell culture system of the invention, the sample port is located in close vicinity of and downstream of the culturing chamber, and such an analysis provides a method to substantially measure the contents of the medium in which a cell is cultured. The method is suited for any type of biological cell, such as mammalian, plant, fungal, insect, or bacterial cells. In a preferred embodiment the cells are mammalian cells, in particular the mammalian cells are cells related to in vitro fertilisation (IVF), and the cells will comprise spermatozoa, unfertilised or fertilised oocytes, and/or embryos. However, as will be obvious to those skilled in the art the bioreactor platform may also be useful for other mammalian cell types, such as stem cells or cells of the immune system, such as monocytes, dendritic cells, T-cells and the like. In a preferred embodiment the mammalian cells are human cells. When the mammalian cells being cultured are intended for being returned to an individual, especially a human being, such as unfertilised or fertilised oocytes for IVF purposes, or stem cells or immune cells for making regenerative medicines or immune therapies, it is important that the sensor does not contact the cells directly.

In addition to these purposes, a mesoscale bioreactor platform as disclosed in the present invention may also be of utility in the culturing of cell types other than mammalian cells. For example, bacterial, yeast, fungal, plant, or insect cells may also be cultured and analysed in the bioreactor platform disclosed herein.

A typical cell culture procedure and the related measuring and analysis of a cell being cultured in a mesoscale bioreactor platform of the invention will initially involve selecting an appropriately designed mesoscale bioreactor platform for the specific type of cells to be cultured. For an IVF-process the mesoscale bioreactor platform will typically have two or more reservoir chambers. Each of these will then be filled with different media representing different growth requirements of the fertilised oocyte during its growth so that the oocyte may be supplemented with an appropriate medium composition at any time during the culturing. Growth media for oocytes are well known within the art, as represented by the culture media available from MediCult A/S (Jyllinge, Denmark).

When the reservoir chambers are filled, an initial medium composition will be applied to the culturing chamber. In one embodiment, the mesoscale bioreactor platform comprises upwards open reservoir and culturing chambers; a layer of a water-immiscible liquid may now be applied to the aqueous liquids in these chambers. Appropriate water-immiscible liquids are oils or fats of a biological source, such as plant oils or the like, or mineral oils or synthetic oils, such as paraffin oil. A transparent water-immiscible liquid is preferred. Paraffin oil is especially preferred. The water-immiscible liquid will form a closure on the chambers, and this will prevent contamination with particles, such as germs or pathogens, prevent evaporation of solvents from the chambers, provide a heat insulating layer. The pH of the aqueous liquids may also be controlled by controlling the pressure of CO₂ above the chambers; the gas above the upwards open chamber may be air or it may be air premixed with e.g. 2-10% CO₂ and/or it may be a trigas with 2-20% O₂.

The temperature of the mesoscale bioreactor platform may now be regulated, e.g. to 37° C., before placing a biological cell, such as a fertilised oocyte, in the culturing chamber. The biological cell will be perfused with a medium composition from one of the reservoirs or from a combination of these as appropriate for the cell. A typical flow-rate will be around 1 μL per hour or higher. When the culturing chamber is perfused with medium from the reservoir chambers this may directly create a flow of liquid from the culturing chamber to the sample port; it may also be necessary to actively pump or otherwise move liquid to the sample port, e.g. by using an integrated pump or by using external pumps. In a preferred embodiment, the bottom of the culturing chamber is transparent so that the cells may be observed microscopically during the culturing. When the flow of liquid to the sample port is established an appropriate sensor may be contacted with the liquid in the container of the sample port, which liquid represents the culturing conditions of the cells in the culturing chamber. Any available sensor may be used in the measurement, and typical parameters of interest in the culturing of a biological cell are pH, conductivity, dissolved oxygen (O₂), carbon dioxide (CO₂), glucose, flow velocity, temperature, and optical density. The sensor may also be capable of measuring a specific parameter, such as an individual nutrient, a vitamin, a metabolite, a signal molecule, a hormone, an enzyme or a protein, such as a cytokine or chemokine. Nucleotides, such as DNA's or RNA's, may also be relevant parameters for measurement with a sensor. Proteins or nucleotides may also be measured ‘in bulk’ where specific entities are not measured individually, e.g. it may be relevant to measure the total concentration of protein, DNA or RNA. Likewise, other types of chemical compounds, e.g. carbohydrates, may also be analysed in bulk.

After contacting the sensor with the liquid in the sample port, the sensor will measure the liquid to obtain an analysis result. The analysis result will provide information regarding the culturing conditions of the cells. Considering the short distance between the culturing chamber and the sample port, this information may be viewed as representing the current conditions of the cell or cells. The analysis result may now be used, by e.g. an operator or through an automated process, as a basis for deciding if any adjustments or modifications to the culturing conditions are necessary. For example, if a value of an operational parameter, such as pH or temperature, is approaching a predefined limit value steps may be taken to keep the value within the predefined limits. If the pH has increased, the CO₂ content above an upwards open culturing chamber may be increased to thereby increase the CO₂ concentration in the medium and thus decrease the pH (or vice versa). The temperature may be modified using a temperature regulating unit containing the mesoscale bioreactor platform. It may also be necessary to modify the chemical composition of the medium supplied to the cell(s) in the culturing chamber. For example, the concentration of a chemical compound, as indicated in the analysis result, may be adjusted by adjusting the composition of media from the different reservoir chambers.

### EXAMPLES

The invention will now be further explained in the following nonlimiting examples.

### Example 1

### Construction of a Mesoscale Bioreactor Platform

A prototype mesoscale bioreactor platform consisting of two layers of substrate materials was designed using the 2D drawing software AutoCAD LT (Autodesk, San Rafael, Calif., USA).

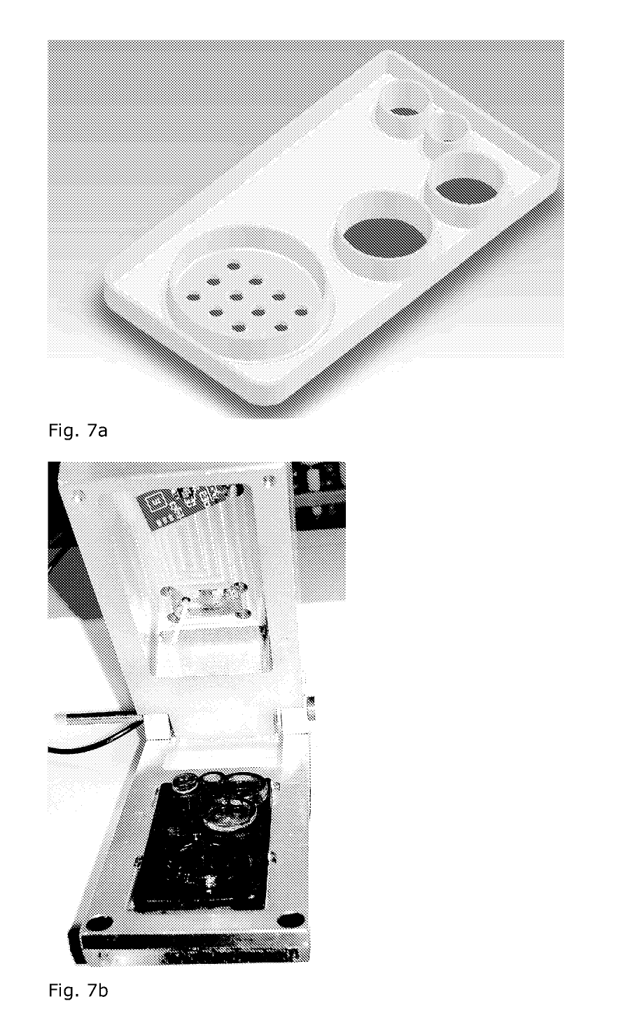

The mesoscale bioreactor platform is illustrated in FIG. 7a and comprised two reservoir chambers 6 mm depth with respective diameters of 14 mm and 12 mm, 12 culture chambers with a diameter of 2.5 mm and a depth of 1.5 mm, as well as another chamber of 7.9 mm diameter and 6 mm depth. These chambers were created in an upper substrate material of black poly(methyl methacrylate) (PMMA) by injection moulding; the total dimensions of the substrate slide was 74×7.4 mm². On the bottom side of the slide channels (of approximately 500 μm diameter) were created using a Synrad Fenix Marker CO₂-laser (Synrad Inc., Mukilteo, Wash., USA). The prepared substrate slide was then laser welded to a transparent PMMA substrate slide of the same size. The transparent PMMA substrate was supplied by Röhm GmbH & Co. (Plexiglas XT20070, Röhm GmbH & Co., Darmstadt, Del.); the layer containing the reservoirs was of approximately 5 mm thickness, all others were of 1.5 mm thickness. Prior to the ablation the AutoCAD LT-designs were converted to encapsulated post-script files and imported into the WinMark Pro software controlling the Synrad Fenix Marker CO₂-laser. Ablation was performed using laser settings which will be well-known to those skilled in the art.

Following an appropriate annealing procedure at 80° C. to prevent stress cracking of the PMMA substrates, the transparent substrates were welded to the bottom surface of the black substrate containing the chambers using a Fisba FLS Iron laser scanner (Fisba Optik AG, St. Gallen, CH) capable of yielding a powerful ˜800 nm laser light. During the welding the substrates were pressurised appropriately using a vice created with glass that is transparent to the laser light. Optimal laser settings for efficient welding are well known within the art.

The welded substrates now defined the channels between the chambers, so that two effluent channels led from each of the reservoir chambers to two separate manifolds. From each manifold a channel led to the first of a series of six culture chambers, so that the two groups each of six culture chambers were connected in parallel with the reservoir chambers.

Each culture chamber had a diameter of 2.5 mm and a depth of 1.5 mm. From each of the last culture chambers in the two series, a channel led to another chamber of 7.9 mm diameter and 6 mm depth. This chamber functions as the coupling means into which a hose may be inserted in order to provide effluent liquid from the culture chambers to an off-platform sample port. The culture chambers are arranged in a 3×4 pattern confined within a 25 mm diameter circle. The substrate defines a wall of 4.5 mm height with an inner surface corresponding to the 25 mm circle. This inner surface further defines a well for a water-immiscible liquid, so that the culture chambers will share a single closure formed by the water-immiscible liquid.

### Example 2

### Construction of a Cell Culture System

Two blocks of aluminium were machined to hold the mesoscale bioreactor platform of Example 1 between the two blocks (of approximately 10×7×3 cm³ size) in an appropriately sized enclosure.

The upper aluminium block was machined to exactly house the bioreactor platform, and a hole (1 mm diameter) was drilled in the upper layer block in a location corresponding to the location of the exit of the mesoscale bioreactor platform. The opening of the hole was expanded to house a rubber O-ring (1 mm ID), and the exit hole fitted with a piece of 0.5 mm ID Teflon tube which was connected to a micro-scale pH-electrode in a compartment in the lower surface of the upper aluminium block so as to define a sample port. The sample port was further connected to a 2 mL syringe pump. The pH-electrode was connected to a sensor board which was further connected to a PC running LabView (ver. 8, National Instruments, Austin, Tex., USA).

The bottom aluminium block was further machined to house a heating coil which was connected to a DC power supply. An electronic temperature sensor was integrated into this aluminium block. The electronic control for the heating element and the temperature sensor were both connected to the sensor board. A custom made LabView application was created to implement a model predictive control (MPC) algorithm for controlling the temperature on the basis of input from the temperature sensor. The two aluminium blocks were attached two each other via a hinge mechanism, so that the mesoscale bioreactor platform could be placed on the temperature regulating element in the lower block. By closing the hinge mechanism the Teflon tube in the upper aluminium block would be inserted into the connection chamber on the mesoscale bioreactor platform so as to create a connection for liquid from the culturing chambers to be led to the sample port.

Control of all pumps was performed from the LabView application via the sensorboard.

The cell culture system thus created is shown in FIG. 7b showing the upper and the lower aluminium blocks in an open position with the mesoscale bioreactor platform inserted; the Teflon tube is visible on the bottom side of the upper aluminium block above the connection chamber.

### Example 3

### Construction of a Cell Culture System

In another design of the cell culture system, the lower aluminium block further comprised a laminar air-flow supply. This consisted of a tube with a horizontal slit (of 1 mm height and 30 mm width) located in a position corresponding to the end of the bioreactor platform with the culturing chambers (as illustrated in FIG. 7) so as to introduce a horizontal laminar air flow above the culturing chamber with a width similar in size to the width of the chamber. The tube had an entry point (located on the outer surface of the upper aluminium block) for connecting to an air supply, e.g. 5% CO₂ in air.

### Example 4

### Use of a Cell Culturing System

A mesoscale bioreactor platform was prepared as outlined in Example 1. The media reservoirs were filled with growth media for blastocyst culture (medium ‘A’ and ‘B’, respectively), and the cell culturing chamber was primed with medium A. A layer of paraffin oil was then applied to each of the upwards open chambers, and the platform was placed in an aluminum housing with a upper block as described in Example 2 and the lower block of Example 3. The temperature of the system was set to 37° C., and a flow of 5% CO₂ in air was applied at approximately 1 L/h to the air supply system to equilibrate the growth media in the bioreactor platform and provide a laminar air flow above the open surfaces of the chambers.

On the following day one fertilised oocyte were placed in each of the culturing chambers, and the cell culturing system was closed. A flow of 7.5 μL/h was provided to the culturing chambers in order to apply fresh medium and remove metabolic waste products. Every 2 hours 15 μL of waste stream was led to the sample port and the pH was measured. The data processing unit, as represented with LabView applications, monitored the progress of the culturing procedure and recorded the pH and temperature. The MPC algorithm was used to control the temperature to 37° C., and a similar algorithm ensured that the pH was kept within 7.25 to 7.45 by adjusting the flow of the CO₂ in air via the air supply.

The cell culturing procedure lasted 3 days, and the composition of medium, i.e. proportion of A and B medium, was controlled according to a predetermined program. In another set-up the cell culturing lasted 5 days.

What is claimed is:

1. A cell culturing system comprising a culturing chamber for culturing a biological cell in a growth medium and a sensor for measuring a signal in the spent growth medium, wherein the culturing chamber is provided in a mesoscale bioreactor platform with an inlet opening for an influent steam of growth medium and a outlet opening for an effluent stream of spent growth medium, said spent growth medium being in fluid communication with a sample port for releasable adoption of the sensor.

2. The cell culturing system according to claim 1, wherein said outlet opening is in fluid communication with an effluent channel provided in the platform.

3. The cell culturing system according to claim 1, wherein the sample port comprises a container having a first opening for the effluent channel from the culturing chamber and a second opening for a waste channel for discharging the spent growth medium.

4. The cell culturing system according to claim 3, wherein the sample port container comprises a third opening for a further inlet channel allowing introduction into the sample port of a liquid different from the effluent stream from the culturing chamber.

5. The cell culturing system according to claim 1, wherein the sensor is capable of measuring pH, conductivity, dissolved oxygen (O₂), carbon dioxide (CO₂), glucose, flow velocity, temperature, and/or optical density.

6. The cell culturing system according to claim 1, wherein the sensor is capable of measuring a nutrient, a vitamin, a signal molecule, a hormone, a metabolite, a protein or an enzyme, and/or a nucleotide, selected from the list consisting of DNA and RNA.

7. The cell culturing system according to claim 1, wherein the sensor is capable of recording an electrical signal, an optical signal, or a fluorescent signal.

8. The cell culturing system according to claim 1, wherein the sample port is integrated on the mesoscale bioreactor platform.

9. The cell culturing system according to claim 2, wherein the effluent channel at one end is connected to the outlet opening of the culturing chamber and at the other end is connected to a coupling device and the sample port is connected to a hose having at the distal end complementary coupling device ensuring transport of spent medium from the effluent channel to the sample port.

10. The cell culturing system according to claim 9, wherein the sample port is external to the mesoscale bioreactor platform.

11. The cell culturing system of claim 9, wherein a sterile filter is present in the stream of spent medium between the effluent channel of the culturing chamber and the sample port.

12. The cell culturing system according to claim 1, comprising two or more chambers for culturing a biological cell, wherein each chamber is in discrete fluid communication with the sample port.

13. The cell culturing system according to claim 12, wherein the two or more chambers for culturing a biological cell are provided in separate bioreactor platforms.

14. The cell culturing system according to claim 9, wherein the bioreactor platform is comprised in a cartridge that fits in the cell culturing system.

15. The cell culturing system according to claim 14, wherein the bioreactor platform is comprised in a compartment comprising a gas supply for creating a laminar air flow around the mesoscale bioreactor platform.

16. The cell culturing system according to claim 1, further comprising a data processing unit configured for analysing a signal from the sensor and converting the signal to an analysis result.

17. The cell culturing system according to claim 16, further comprising a control to control operational parameters in the culture chamber, wherein the data processing unit is configured for sending commands to a control unit or regulating the control to control operational parameters.

18. A method of measuring an effluent stream of spent growth medium from a culturing chamber in a cell culturing system according to claim 1, comprising the steps of:
providing a biological cell in the culturing chamber,
perfusing the culturing chamber with a growth medium entering through the inlet opening of the culturing chamber and exiting through the outlet opening,
conveying the spent growth medium to a sample port;
contacting the spent growth medium of sample port with the sensor, and
measuring a signal in the spent growth medium.

19. A method according to claim 18, further comprising the step of adjusting the conditions in the culturing chamber on the basis of the measured signal.

20. A method according to claim 18, wherein the biological cell is a mammalian cell, selected from the list consisting of a spermatozoon, an oocyte, an embryo, a stem cell, a monocyte, a dendritic cell, and a T-cell.
